(19) United States
(12) Patent Application Publication
(10) Pub. No.: US 2004/0153076 A1
(43) Pub. Date: Aug. 5, 2004

(54) Ligament graft cage fixation device

(76) Inventors: Wamis Singhatat, Santa Ana, CA (US); Hugh S. West JR., Sandy, UT (US)

Correspondence Address: GENE WARZECHA, LINVATEC CORPORATION, 11311 CONCEPT BOULEVARD, LARGO, FL, 33773

(21) Appl. No.: 10/695,535

(22) Filed: Oct. 28, 2003

Related U.S. Application Data
(60) Provisional application No. 60/421,706, filed on Oct. 29, 2002.

(51) Int. Cl. A61B 17/86; A61F 2/46; A61B 17/88; A61F 2/08

(52) U.S. Cl. 606/73; 623/13.14; 606/104

(57) ABSTRACT

A graft retaining implant is provided for retaining a graft in a bone tunnel. Instruments and methods are also provided for use with the graft retaining implant.

### CROSS-REFERENCE TO RELATED APPLICATIONS

This application claims the benefit of U.S. Provisional Application No. 60/421,706, filed Oct. 29, 2002.

### FIELD OF THE INVENTION

The invention relates to soft tissue repair and reconstruction. More particularly, the invention relates to the fixation of a graft within a bone tunnel.

### DESCRIPTION OF THE PRIOR ART

The repair and reconstruction of torn or damaged soft tissues is a common surgical procedure. For example, replacement graft ligaments may be secured at the site of the original ligament. The procedure generally involves drilling bone tunnels into adjacent bones at the site of the original ligament and securing within these bone tunnels a graft ligament. In many applications, such as in the knee joint, such procedures may be performed arthroscopically. The graft ligament may be an autograft, an allograft, a xenograft, and/or it may be totally artificial and synthetic. The most common types of graft ligaments include ones which may be bone-tendon-bone or soft tissue (such as semitendinosus and gracilis tendons), both types harvested by techniques well known to those skilled in the art.

For example, repair of the anterior cruciate ligament (ACL) of the knee is often performed arthroscopically in a procedure which involves drilling a bone tunnel through the proximal tibia and into the distal femur. A variety of different types of graft ligaments may be secured in the bone tunnels in the femur and the tibia to replace the ACL.

In order to perform the procedure arthroscopically, various fixation methods are used to secure the graft ligament within the femur and within the tibia. The fixation method should be able to satisfactorily engage the bone in the wall of the bone tunnel or on the cortical bone surface. Consideration must be given to the fact that the bone may have only a thin layer of relatively hard cortical bone, such as in the anterior proximal surface and tibial plateau of the tibia. The bone may be otherwise relatively soft, cancellous bone. Depending upon the patient, the quality of the bone may vary considerably, particularly the cancellous bone. It would, therefore, be desirable to have a device capable of achieving fixation of the graft while eliminating the variability of fixation caused by the varying strength and density of cancellous bone.

Another consideration is that for biological graft fixation, that is, fixation resulting from tissue growing between the bone tunnel wall and the graft, some consideration should be given to facilitating such growth by enabling direct contact between the graft and the surrounding bone.

### SUMMARY

The present invention provides a graft retaining implant for retaining a graft in a bone tunnel formed in a bone. Instruments and methods are also provided for use with the graft retaining implant.

In one aspect of the invention, a ligament fixation implant includes a frame having a plurality of elongated members situated in longitudinal alignment with an axis. A plurality of rings is connected to each of the elongated members and is aligned transverse to the elongated members.

In another aspect of the invention, a graft retaining system includes means for engaging a bone and means for engaging the graft. The means for engaging the bone includes a first member having a first portion that engages the bone in positive axially fixed relationship and a second portion including a plurality of longitudinally spaced closed rings positionable within the bone tunnel around the graft. The means for engaging the graft includes a second member insertable through the rings adjacent the graft to press the graft radially outwardly such that it bulges over the rings to fix the graft axially within the bone tunnel.

In another aspect of the invention, a graft fixation implant includes a cage and an interference screw. The cage has at least one elongated member situated in longitudinal alignment with an axis and at least one ring connected to it. The interference screw is sized to be inserted axially through the ring to compress the graft ligament against the cage and the bone tunnel wall.

In another aspect of the invention, a graft fixation implant for securing a graft in a bone tunnel includes a cage body having a lumen for receiving a portion of the graft. The implant further includes antirotation means for positively engaging the inner tunnel wall to resist rotation of the cage about the bone tunnel axis. The implant further comprises means for compressing the graft radially outwardly into engagement with the cage body and inner tunnel wall.

In another aspect of the invention, an instrument for inserting a graft cage with a plurality of rib members includes means for engaging the cage rib members to brace them against radially inward deflection. The instrument further includes means for imparting an axial insertion force on the cage body.

In another aspect of the invention, an instrument for preparing a bone tunnel includes a shaft having a proximal end, a distal leading end, and an enlarged portion proximal to the distal leading end. The enlarged portion forms an enlarged counterbore in the bone tunnel upon being inserted into the bone tunnel

In another aspect of the invention, a method for retaining a graft in a bone includes forming a tunnel in the bone; inserting a first member into the bone tunnel, the first member having a plurality of longitudinally spaced closed rings; inserting the graft through the rings; and inserting a second member adjacent the graft to press the graft radially outwardly to cause it to bulge around the rings to fix the graft axially within the bone.

In another aspect of the invention, a method of securing a graft ligament in a bone tunnel includes providing a cage comprising at least one elongated member situated in longitudinal alignment with an axis, at least one ring connected to the at least one elongated member; forming a bone tunnel; inserting the cage into the bone tunnel; inserting an end of a graft axially through the ring; providing an interference screw; and inserting the interference screw axially through the ring to compress the graft ligament against the ligament fixation implant and the bone tunnel wall.

In another aspect of the invention, a method of securing a graft ligament in a bone tunnel having an inner tunnel wall includes providing a cage including antirotation means for positively engaging the inner tunnel wall to resist rotation of the cage about the bone tunnel axis; forming a bone tunnel; inserting the cage into the bone tunnel with the antirotation means engaging the inner tunnel wall; inserting an end of a graft axially through the cage; providing an interference screw; and inserting the interference screw axially through the cage to compress the graft ligament against the cage and the bone tunnel wall.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is a perspective view of a human knee joint showing the insertion of an illustrative graft and graft fixation device according to the present invention.

FIG. 2 is a perspective view of the illustrative graft cage of FIG. 1.

FIG. 3 is a side plan view of the illustrative graft cage of FIG. 1.

FIG. 4 is an end plan view of the illustrative graft cage of FIG. 1.

FIG. 5 is a perspective view of an illustrative tunnel preparation instrument for use with the illustrative graft cage of FIG. 1.

FIG. 6 is a section view taken along line 6-6 of FIG. 1 showing the bone tunnel prepared for the illustrative graft cage of FIG. 1.

FIG. 7 is an end plan view showing the bone tunnel of FIG. 6.

FIG. 8 is a section view showing the illustrative graft cage of FIG. 1 installed in the tunnel of FIG. 6.

FIG. 9 is an end plan view of the illustrative graft cage of FIG. 1 installed in the tunnel of FIG. 6.

FIG. 10 is a section view showing the illustrative graft cage of FIG. 1 installed in the tunnel of FIG. 6 and a graft installed through the cage.

FIG. 11 is a section view showing the illustrative graft cage of FIG. 1 installed in the tunnel of FIG. 6 and a graft installed through the cage and secured with a bone screw.

FIG. 12 is an end plan view showing the illustrative graft cage of FIG. 1 installed in the tunnel of FIG. 6 and a graft installed through the cage and secured with a bone screw.

FIG. 13 is a section view taken along line 13-13 of FIG. 1 showing the graft cage of FIG. 1 installed in the bone tunnel.

FIG. 14 is a section view similar to FIG. 13 showing a graft cage similar to that of FIG. 1 having optional angled prongs.

FIG. 15 is a section view similar to FIG. 13 showing a graft cage similar to that of FIG. 1 having an optional angled flange.

FIG. 16 is a perspective view of the graft cage of FIG. 1 having optional helical rings and an optional cage clip.

FIG. 17 is a side plan view of the graft cage of FIG. 16 shown with a screw.

FIG. 18 is a side plan view of the graft cage of FIG. 16 shown with an alternative fixation screw.

FIG. 19 is a perspective view of the graft cage of FIG. 16 having optional screw threads formed at the end of the cage to receive a screw.

FIG. 20 is a side plan view of an instrument including a handle and an obturator shown mated with a graft cage.

FIG. 21 is a perspective view of the handle of FIG. 19.

FIG. 22 is an end plan view of the graft cage of FIG. 19 showing an optional pair of lips for coupling the graft cage to the handle.

FIG. 23 is a perspective view of the obturator of FIG. 19.

### DESCRIPTION OF THE ILLUSTRATED EMBODIMENTS

The graft fixation system of the present invention may be used to attach any appropriate graft including, for example, supplemental and/or replacement grafts for the soft tissues associated with the skeletal system. For example, the system may be used to replace soft tissues associated with skeletal joints such as the hip, knee, shoulder, wrist, elbow, ankle, vertebral, phalangeal, temporomandibular, and other joints and locations within a body. For example, the graft fixation system may be used to attach, within a bone tunnel, grafts associated with human knee joint tissues such as the anterior cruciate ligament, the posterior cruciate ligament, the medial collateral ligament, and the lateral collateral ligament. In the illustrative embodiments, a graft fixation system is depicted for use in securing an anterior cruciate ligament graft within a tibial bone tunnel. It will be understood by those skilled in the art that this invention may be suitable for other applications as well.

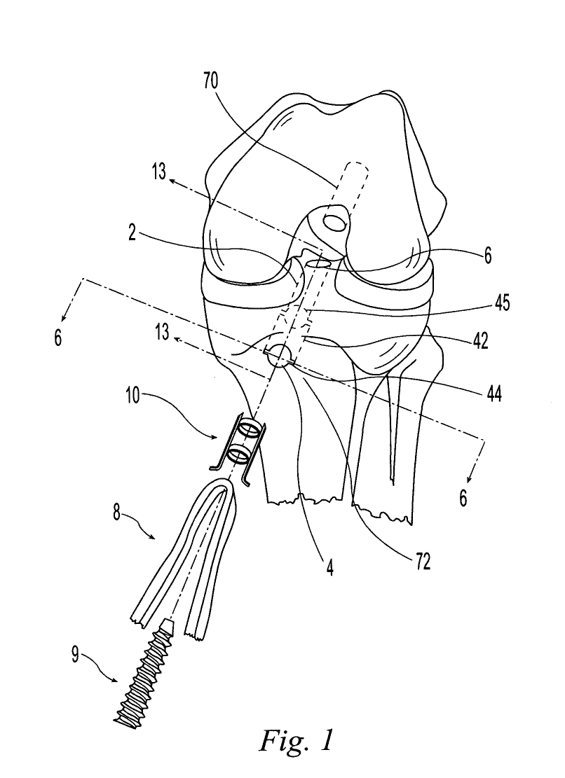

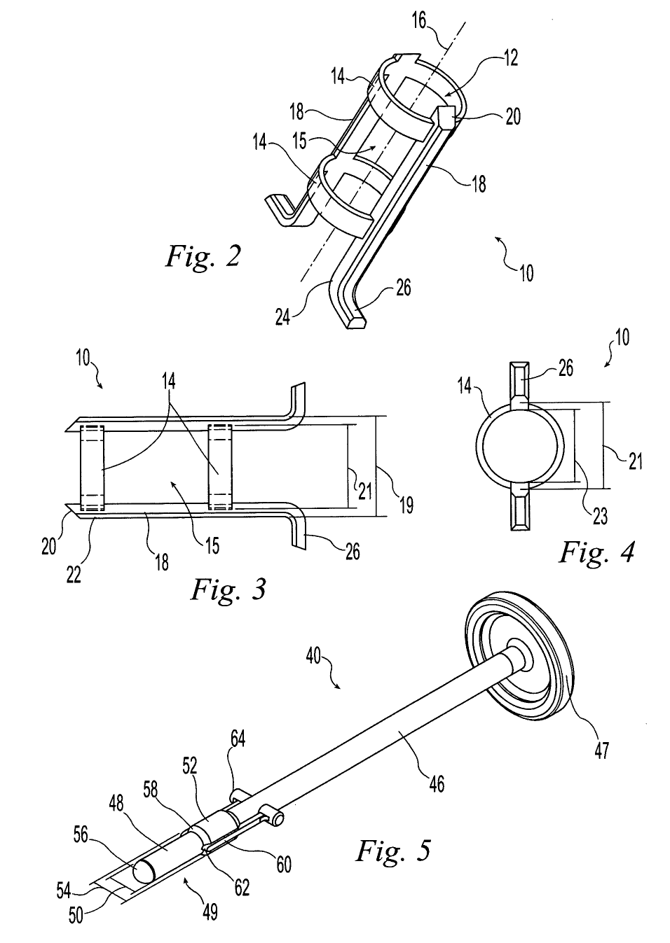

FIGS. 1-4 depict an illustrative embodiment of the invention constructed in the form of a tubular graft frame or cage 10 for insertion into a tibial tunnel 2 having a proximal end 4 and a distal end 6. The terms “proximal” and “distal” are intended to refer to the orientation of the graft cage 10 within its bone tunnel, the proximal end being closer to the tunnel entrance and the distal end being farther away. A graft 8 is anchored in the cage 10 with a screw 9. The cage 10 serves to reinforce the tunnel 2 wall to facilitate anchoring the graft 8 adjacent the tunnel 2 wall. The cage 10 also engages the bone in axially fixed relationship to fix the axial position of the graft 8 relative to the bone tunnel 2. The cage 10 includes an annular body defining a lumen 12 axially aligned along an axis 16 and an elongated support surface. In the illustrative embodiment, the annular body includes a pair of longitudinally spaced circular rings 14 disposed along its axis 16 and the elongated support surface includes a pair of diametrically opposed axial ribs 18 that connect the rings 14. The rings 14 are closed and therefore have relatively high hoop strength to resist radially outward forces. The ribs 18 maintain the rings 14 in fixed axial spacing so that a gap 15 is maintained between the rings 14. The ribs 18 include a taper 20 at their distal ends 22 to facilitate insertion and a cortex engaging means at their proximal ends 24. In the illustrative embodiment, the cortex engaging means includes radially outwardly extending cortical prongs 26 for engaging the anterior tibial cortex 72. The prongs 26 maintain the cage 10 in axially fixed position within the tibial tunnel 2. The axial ribs 18 protrude a predetermined distance 19 radially beyond the outer diameter 21 of the rings to engage the bone adjacent the inner tunnel wall of the tibial tunnel 2 and resist rotation of the cage within the tibial tunnel 2. The cage 10 may be made in various diameters and lengths. In the illustrative embodiment, the cage 10 is sized such that its inner diameter 23 matches that of the distal end 6 of the tibial tunnel 2. The diameter of the graft 8 should be the same as or less than the internal diameter 23 of the rings 14 to allow the graft 8 to be passed through the cage 10 and tunnel 2. The graft 8 may need to be compressed to fit through the cage 10 and tunnel 2.

The cage 10 may be a unitary or multi-piece construction including any suitable biocompatible materials. Exemplary materials include metals, polymers, and/or other suitable materials and combinations thereof. For example, the cage 10 may include metals including stainless steels, titanium, titanium alloys, cobalt-chromium steels, nickel-titanium alloys, and/or others. The cage 10 may include nonresorbable polymers including polyolefins, polyesters, polyimides, polyamides, polyacrylates, poly(ketones), fluropolymers, siloxane based polymers, and/or others. The cage 10 may include resorbable polymers including polyesters (e.g. lactide and glycolide), polyanhydrides, poly(aminoacid) polymers (e.g. tyrosine based polymers), and/or others. The cage 10 may include other materials including nonresorbable and resorbable ceramics (e.g. hydroxyapitite, calcium sulfate) or biocompatible glasses. The illustrative cage 10 may be constructed by machining, punching, welding, molding, sintering, and/or other suitable methods. For example, a suitable cage 10 may be injection molded from a resorbable polymer.

The principles embodied by the invention include the provision of a fixed and predictable surface against which the graft 8 may be compressed and the provision of a means by which the graft 8 and the surrounding bone may grow together. These principles are achieved by the illustrative embodiments disclosed herein, however, it will be understood that other modifications can be made which will also be suitable. For example, a cage 10 having any number of ribs 18 and any number of rings 14 will fall within the scope of the invention. Additionally, a cage 10 formed of a perforated cylindrical wall would also be suitable. Also, a bioabsorbable non-perforated cylinder could be used if its degradation time was sufficient to foster growth between the graft and the bone. Additionally, it can be appreciated that the shape of the cage 10 can be non-cylindrical (i.e. ellipsoidal, rectilinear, or other symmetrical or asymmetrical shape or geometry).

Another significant parameter of the graft cage 10 is its relatively high hoop strength. That is, the rings 14 are rigid and resist expansion to provide a surface against which a graft 8 may be compressed. Thus, the connection between the graft 8 and graft cage10 will be reliable regardless of the strength or density of the cancellous bone surrounding the cage 10. Given this, once the cage 10 is secured adjacent the cortical bone at the proximal end 4 of the tibial tunnel 2, a strong and reliable ACL fixation will exist.

The tibial tunnel 2 is prepared to receive the graft cage 10 with a tunnel dilator 40. The dilator 40 forms a counterbore 42 and axial channels 44 adjacent the proximal end 4 of the tibial tunnel 2. The dilator 40 includes a cylindrical body 46 having a working end 49 for insertion into the tibial tunnel 2 and an input end 47 for being impacted to drive the working end 49 into the tibial bone tunnel 2. The working end 49 includes a distal portion 48 with a predetermined diameter 50 and a larger proximal portion 52 with a predetermined diameter 54. The distal portion 48 is sized to fit within the tibial tunnel 2. A bullet shaped tip 56 aids insertion of the distal portion 48. The proximal portion 52 is sized to compress the tibial tunnel 2 wall to form the enlarged counterbore 42 to receive the cage 10 rings 14. A transition taper 58 blends the distal portion 48 and proximal portion 52. The dilator 40 further includes channel forming means to create channels 44 to receive the ribs 18 of the graft cage 10. In the illustrative embodiment, the channel forming means includes a pair of diametrically opposed rails 60 with outer walls spaced apart by the same distance 19, or slightly further, than the ribs 18 protrude beyond the rings 14. Each rail 60 has an elongated body with a tapered leading edge 62. A depth stop 64 projects from the dilator 40 adjacent the trailing end of the rails 60 to limit the insertion depth of the dilator 40. In the illustrative embodiment, the dilator 40 includes two rails 60 to correspond to the graft cage which has two ribs 18. However, any number of rails 60 and ribs 18 may be included.

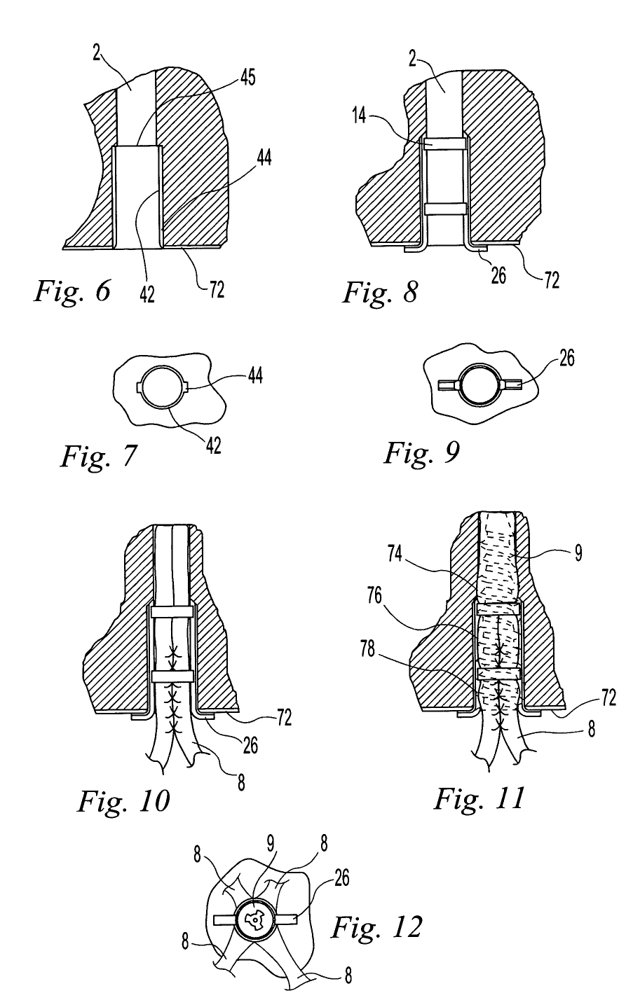

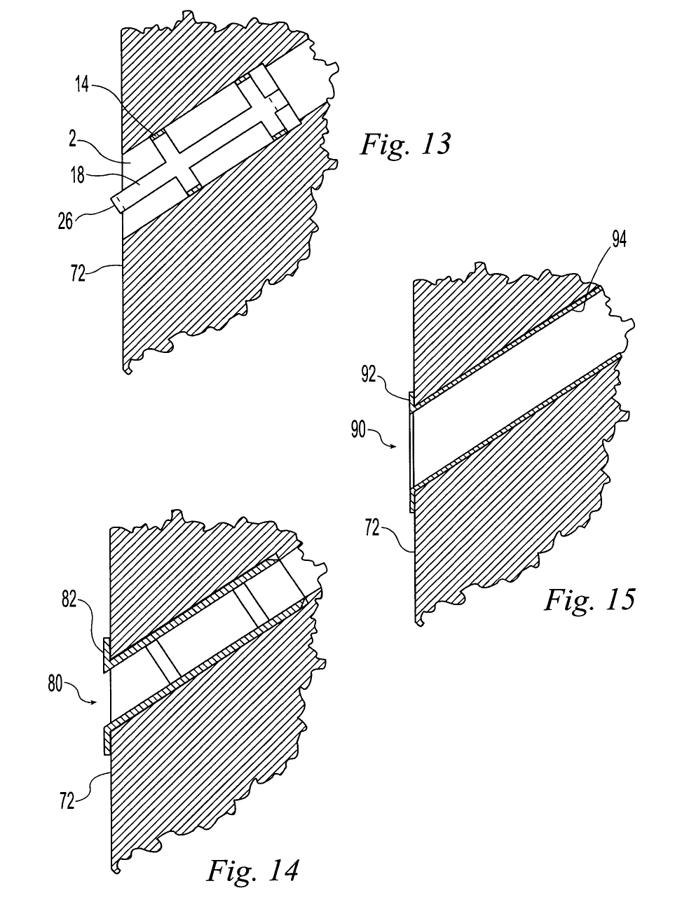

Referring now to FIGS. 6-12, a method is described for using the graft cage 10 to secure the graft 8. The illustrative graft cage 10 is shown in use with a soft tissue graft 8 as opposed to a bone-tendon-bone graft. The soft tissue graft 8, e.g. a semitendinosus and/or gracilis graft, is harvested and prepared outside the body in a known manner. This preparation may include whipstitching as shown and/or other preparation to ready it for use. A femoral tunnel 70 (as shown in FIG. 1) and a tibial tunnel 2 are drilled in a typical fashion and are sized to match the graft 8 diameter. The method of using the invention will be described with reference to the tibial tunnel 2; specifically the proximal end 4 of the tibial tunnel 2 adjacent the anterior tibial cortex 72.

As shown in FIGS. 6 and 7, a counterbore 42 and diametrically opposed channels 44 are created in the tibial tunnel 2 by impacting the dilator 40 into the proximal end 4 of the tibial tunnel 2. The counterbore 42 is formed by the proximal portion 52 of the dilator 40 while the channels 44 are formed by the rails 60.

The graft cage 10 is oriented such that the ribs 18 of the cage 10 seat into the channels 44 in the tibial tunnel 2. The graft cage 10 is fully inserted until the prongs 26 of the cage 10 rest on the anterior cortical surface 72 as shown in FIGS. 8 and 9. The engagement of the prongs 26 with the anterior cortical surface 72 positively fixes the graft cage 10 axially relative to the tunnel. In addition to the prong 26 engagement, the distal most ring 14 of the graft cage can seat on the shoulder 45 formed by the counterbore 42 to positively fix the graft cage 10 axially. The prong 26 on cortex 72 engagement and the ring 14 on shoulder 45 engagement can be used together or either can be used alone. As shown in FIG. 10, a graft 8 is then passed through the lumen 12 of the cage 10 and the tibial 2 and femoral 70 tunnels and fixed on the femoral side in a conventional manner.

The graft 8 is fixed on the tibial side by the graft cage 10 and fixating member. The fixating member is placed within the lumen 12 of the cage 10 to compress the graft 8 radially outwardly into engagement with the cage 10 rings 14 and ribs 18 and the bone tunnel 2 wall. The shape of the ring 14 edges may be adjusted to control how aggressively the rings 14 grip the graft 8. After the graft 8 is placed within the cage 10 as shown in FIG. 10, the graft 8 is tensioned appropriately and the fixation member is inserted into the lumen of the cage 10 and secured in place. It may be inserted either centrally or eccentrically to the bundles of the graft. In the illustrative embodiment, an interference screw 9 having a headless, threaded body is used as a fixating member. The engagement of the cage ribs 18 with the channels 44 formed adjacent the bone tunnel 2 prevents rotation of the cage 10 during insertion of the screw 9. Alternatively, other screw forms, e.g. headed screws, or other fasteners, e.g. pins or wedges having smooth, ribbed or otherwise contoured surfaces, may be used. The interference screw 9 used in the illustrative embodiment may have a conventional driver interface to allow insertion with a screw driver, and may have a constant diameter along most of its threaded body. The diameter of the body of the screw 9 is sized such that it allows insertion of the screw 9 into the lumen 12 of the cage 10 after placing the graft 8 into the cage 10 and provides compression of the graft 8 radially outwardly against the inner surface of the cage 10 members and the bone. As seen in FIG. 11, the screw 9 causes the graft 8 to bulge outwardly and grip the rings 14. The graft 8 bulges outwardly to form a distal bulge 74 distal to the rings 14, an intermediate bulge 76 between the rings 14, and a proximal bulge 78 proximal to the rings 14. The rings 14 are positioned distal to the proximal end 24 of the cage 10 to allow for the proximal bulge 78 within the tunnel 2 and the additional axial fixation strength that it contributes. These bulges 74, 76, 78 contact the tunnel 2 wall and facilitate direct fixation of the graft 8 to the bone. The intermediate 76 and proximal bulges 78 also cause the graft 8 to grip the rings 14 to resist distal axial displacement of the graft 8 within the tunnel 2. The cage 10 may be provided with more than two rings 14 in which case the screw 9 will cause the graft 8 to bulge in more locations to grip the additional rings. In the illustrative embodiment, the screw 9 directly contacts the graft 8 such that the screw 9 threads grip the graft 8.

The interference screw 9 may be made of conventional materials such as biocompatible metals, polymers, ceramics and/or bioabsorbable materials such as PLLA or other suitable materials as discussed relative to the graft cage 10.

If the ribs 18 are made relatively narrow as shown, that is, subtending an arc about the axis 16 of less than approximately 20°, the cage 10 may be inserted into a tibial tunnel 2 that is angled relative to the cortical surface 72 without the prongs 26 protruding excessively from the tunnel 2 as shown in FIG. 13. Such a cage is symmetric and can be rotated 180° without changing its fit to the bone.

Alternatively, if the ribs are made to subtend a large arcuate portion, or if they terminate in an annular proximal ring, the plane of the prongs or the proximal ring may be inclined relative to the longitudinal axis of the cage to prevent the prongs or ring from protruding excessively. For example, prongs 26 oriented like those of FIG. 13 may be inclined upwardly so that they lie flat against the bone at the 3 and 9 o'clock positions of the tunnel opening. Alternatively, for example, the prongs can be oriented as in FIG. 14 which shows a cage 80 having prongs 82 that are angled to lie flush adjacent the tibial cortex 72 at the 6 and 12 o'clock positions. The prongs 82 may be angled to fit the bone at other orientations as well. In another example, FIG. 15 shows a cage 90 having an annular proximal ring 92. The ring 92 is angled to lie flush adjacent the tibial cortex 72. In this example, the cage 10 body includes a solid cylindrical wall 94. The wall 94 may be perforated (not shown) and/or may be porous, fenestrated, and/or biodegradable, with or without bone growth inducing properties.

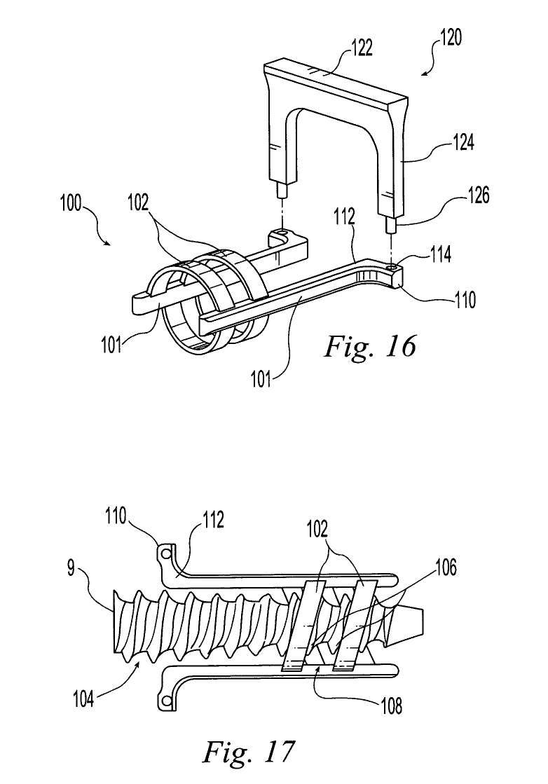

FIGS. 16-19 show another example of the invention. In this example, the graft cage 100 includes elongated ribs 101 connecting rings 102 that are transversely helical rather than cylindrical. The cage ring 102 helix angle corresponds to the screw thread 104 helix angle. When the screw 9 is threaded into the graft 8/cage 100 assembly, the lands 106 of the screw thread 104 will align with the space 108 between the rings 102 and more uniformly compress the graft 8 material between the rings 102. The graft 8 also is trapped between the rings 102 and screw threads 104. The ribs 101 form prongs 110 that project radially outwardly at their proximal ends 112. The ribs 101 include an engagement mechanism adjacent their proximal ends 112. In the illustrative example, the engagement mechanism includes holes 114 formed in the prongs 110. The cage 100 also includes a removable clip 120 having a generally 'U”-shaped body including a crossbar 122 and legs 124. The legs 124 terminate in engagement members for engaging with the proximal end of the ribs 101. In the illustrative example, the engagement members include cylindrical stubs 126 engageable with the holes 114 formed in the prongs 110. In use, the clip 120 is engaged with the graft cage 100 and used as a handle to facilitate manipulating the graft cage 100 and applying an axial insertion force to the graft cage 100. The clip 120 also acts to brace the cage 100 to maintain the spacing between the proximal ends 112 of the ribs 101 to keep the prongs 110 from bending or deflecting radially inwardly. In this way, the prongs 110 maintain their grip on the anterior tibial cortex until the screw 9 is placed to firmly fix the assembly in place. Once the screw 9 is inserted, the clip 120 is removed.

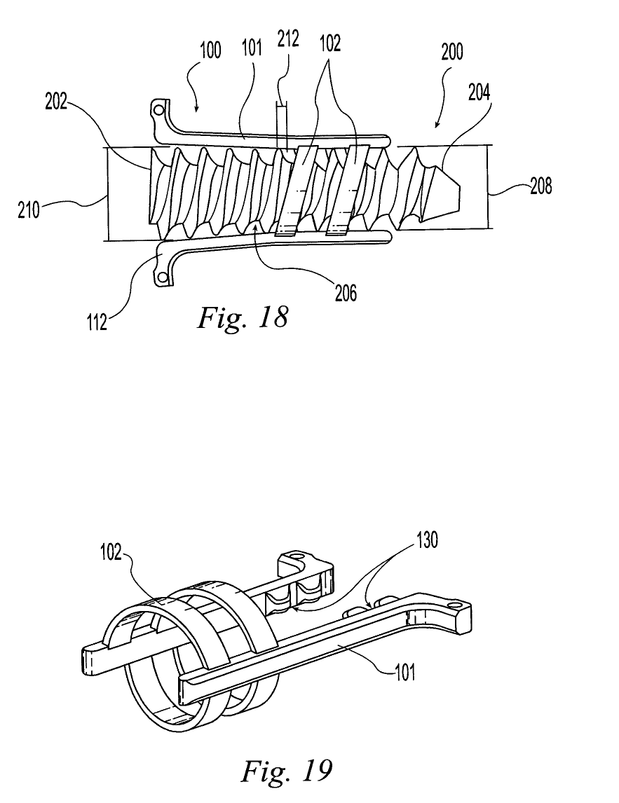

FIG. 18 shows an optional screw 200 for use with the graft cage 100. The screw 200 includes a proximal end 202, a distal end 204, and a screw thread 206 spiraling between the ends. The screw thread 206 has a distal major diameter 208 adjacent the distal end 204 sized to fit within the rings 102. In the illustrative embodiment, the distal major diameter 208 is a close sliding fit within the rings 102. The screw thread 206 has a proximal major diameter 210 adjacent the proximal end 202. In the illustrative embodiment, the proximal major diameter 210 is larger than the distal major diameter 208 and forces the ribs 101 to deflect radially outwardly near the proximal end 202 upon insertion of the screw 200. A relatively short transition region 212 lies between the distal and proximal major diameters 208, 210. When the screw 200 is inserted between the graft 8 bundles within the cage 100, the graft 8 bulges outwardly to form a distal bulge distal to the rings 102, an intermediate bulge between the rings 102, and a proximal bulge proximal to the rings 102. The larger proximal major diameter 210 of the screw thread 206 causes the graft 8 to bulge out even further and overlie the proximal most ring. The larger proximal major diameter 210 also presses against the ribs 101 and causes them to deflect outwardly near the proximal end 112 so that they tightly grip the anterior cortex 72 of the tibia.

FIG. 19 shows the graft cage 100 of FIG. 16 with optional cage threads 130 formed adjacent the proximal ends 112 of the ribs 101. The cage threads 130 engage the threads of the screw 9 so that as the screw advances into the rings 102, the screw 9 and ring 102 helixes will be aligned.

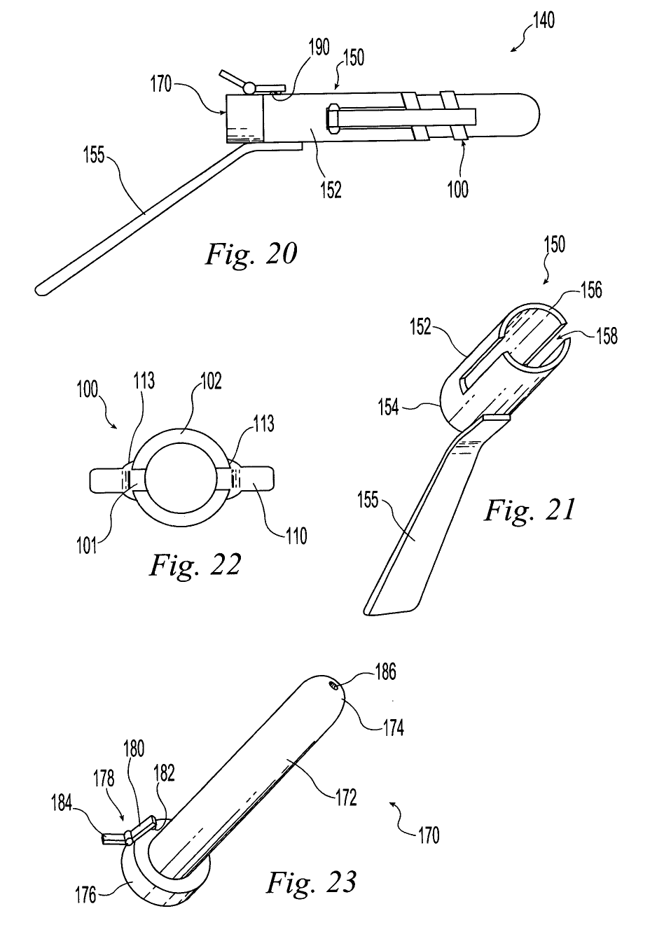

FIGS. 20-23 depict an optional instrument for use with the graft cages of FIGS. 1-19. An inserter assembly 140 for gripping and inserting the graft cage 10 includes a handle 150 and an obturator 170. The inserter 140 provides both the gripping and bracing functions of the clip 120 of FIG. 16 as well as additional functionality. The handle 150, includes a tubular body 152 having a proximal end 154 and a distal 156 end. A grip 155 projects at an angle from the body 152. The inner and outer diameters of the body 152 match the inner and outer diameters of the graft cage rings 102. The body 152 includes diametrically opposed longitudinal slots 158 from the distal end 156 toward the proximal end 154. The slots 158 are sized to receive the ribs 101 of the graft cage 100. For use with a graft cage 100 having helical rings 102, as shown, the distal end 156 of the tubular body 152 is formed at an angle corresponding to the ring 102 helix angle. In use, the graft cage ribs 101 are slid into the longitudinal slots 158 until the rings 102 abut the distal end 156 of the body 152. The distal ends of the ribs 101 project distally away from the body 152. To further stabilize the cage 100 on the inserter 140, the cage 100 may be formed with optional lips 113 that project circumferentially from the prongs 110. The lips 113 engage the outer wall of the inserter body 152 to keep the prongs 110 from bending or deflecting radially inwardly and to keep the cage 100 from slipping from side to side on the inserter 140.

An optional obturator 170 may be used to stabilize the cage 100 on the inserter 140 and to provide a leading tip to facilitate inserting the cage 100 into a bone tunnel. The obturator includes an elongated cylindrical body 172 having a distal end forming a bullet shaped tip 174 and a proximal end having a depth limiting flange 176. The body 172 is sized for a sliding fit within the handle body 152. The obturator may further include a latch 178 having an engagement portion and an actuating portion. In the illustrative example, the latch 178 is pivotably mounted to the flange 176 and includes a distally extending engagement arm 180 with an engagement lug 182 extending radially inwardly from the arm 180. An actuating arm 184 connects to the engagement arm 180 and extends proximally. A spring (not shown) biases the engagement arm 180 toward the obturator body 172. Pressing the actuating arm 184 overcomes the spring force and moves the engagement arm 180 away from the obturator body 172. The obturator may further include a longitudinal bore 186 to permit it to be inserted over a guide wire.

In use, the obturator 170 is inserted into the handle 150 until the flange 176 abuts the proximal end 154 of the handle body 152. The engagement lug 182 of the latch 178 engages an engagement lug 190 on the handle body 152 to retain the obturator in place. The graft cage 100 is slid over the obturator and into engagement with the handle as described above. The graft cage 100/inserter 140 assembly is then directed toward the bone tunnel 2. The bullet end 174 of the obturator 170 is engaged with the tunnel 2 entrance and the graft cage 100 and inserter assembly 140 are slid into the tunnel 2. The obturator 170 is withdrawn to permit access to the cage 100 interior and the bone tunnel 2. The handle 150 is held by the user to maintain the graft cage 100 in position while the graft 8 is inserted through the handle 150, through the graft cage 100, and through the bone tunnels. Once the graft 8 is positioned, the handle 150 is withdrawn and the fixation member, e.g. the screw 9, is inserted to fix the graft 8 and cage 100 in place.

It will be understood by those skilled in the art that numerous improvements and modifications may be made to the preferred embodiment of the invention disclosed herein without departing from the spirit and scope thereof.

What is claimed is:

1. A ligament fixation implant comprising:
a frame comprising a plurality of elongated members situated in longitudinal alignment with an axis;
a plurality of rings connected to each of the elongated members and aligned transversely to the elongated members.

2. The ligament fixation implant of claim 1 wherein the elongated members have a distal end and a proximal end and further comprising a radially outwardly extending projection secured to the proximal end of each of the elongated members.

3. The ligament fixation implant of claim 2 wherein the distal end of each of the elongated members is tapered.

4. The ligament fixation implant of claim 2 wherein each of the plurality of rings is connected to each of the plurality of elongated members such that the rings are held together in a fixed longitudinal spacing along the axis.

5. The ligament fixation implant of claim 4 wherein the rings are each spaced a predetermined respective distance from the proximal end of the elongated members.

6. The ligament fixation implant of claim 1 wherein each of the plurality of rings is aligned in a plane perpendicular to the elongated members.

7. The ligament fixation implant of claim 1 wherein each of the plurality of rings forms a closed loop.

8. The ligament fixation implant of claim 1 further comprising an interference screw having a body with a proximal end, a distal end, and a longitudinal axis therebetween, a helical thread extending radially from the screw body, the helical thread having a distal major diameter adjacent the distal end and a proximal major diameter adjacent the proximal end, the proximal major diameter being greater than the distal major diameter, the screw able to be inserted axially through the rings along with a ligament to cause the ligament to bulge radially outwardly and grip the rings.

9. The ligament fixation implant of claim 8 wherein the screw causes the ligament to bulge more proximally than distally.

10. The ligament fixation implant of claim 8 wherein the screw thread distal major diameter is sufficiently small to pass through the rings and the screw thread proximal major diameter is too large to pass through the rings.

11. The ligament fixation implant of claim 8 wherein the elongated members have a distal end and a proximal end and the rings are each spaced a predetermined respective distance from the proximal end of the elongated members, the screw thread proximal major diameter being substantially contained in the space between the proximal end of the elongated members and the rings when the screw is fully inserted.

12. The ligament fixation implant of claim 11 wherein the screw further causes the proximal ends of the elongated members to deflect outwardly to tightly engage the bone.

13. A graft retaining system for retaining a graft in a bone, the bone containing a bone tunnel having a longitudinal axis, a tunnel opening, an internal tunnel wall, and an external bone surface adjacent the tunnel opening, the system comprising:
means for engaging the bone including a first member having a first portion that engages the bone in positive axially fixed relationship and a second portion including a plurality of longitudinally spaced closed rings positionable within the bone tunnel around the graft; and
means for engaging the graft including a second member insertable through the rings adjacent the graft to press the graft radially outwardly such that it bulges over the rings to fix the graft axially within the bone tunnel.

14. The graft retaining system of claim 13 further comprising a removable bracing member engageable with the plurality of members to temporarily brace them against radially inward deflection.

15. The graft retaining system of claim 13 wherein the means for engaging the bone comprises a plurality of hook members extending outwardly from the tunnel opening to engage the external bone surface adjacent the tunnel opening.

16. The graft retaining system of claim 13 wherein the means for engaging the bone comprises a radially projecting annular ring.

17. The graft retaining system of claim 13 wherein the means for engaging the bone comprises a surface engageable with a shoulder formed in a countersink within the bone tunnel.

18. The graft retaining system of claim 13 wherein the first member further comprises a third portion that engages the bone to prevent rotation of the first member about the tunnel axis.

19. The graft retaining system of claim 13 wherein the first member further comprises at least one longitudinal rib connecting the plurality of rings, the at least one rib extending radially outwardly beyond the rings a predetermined distance, the at least one rib being receivable by a channel formed longitudinally in the internal tunnel wall.

20. The graft retaining system of claim 13 wherein the second member comprises a screw having a helical thread with a helix angle and the closed rings are angled to form a helix with a helix angle corresponding to the screw helix angle.

21. The graft retaining system of claim 13 wherein the means for engaging the bone comprises a plurality of generally parallel elongated members having a proximal end and a distal end, a plurality of rings being connected to each of the elongated members and aligned transversely to the elongated members, the means for engaging the graft comprising an interference screw having a body with a proximal end, a distal end, and a longitudinal axis therebetween, a helical thread extending radially from the screw body such that the screw may be inserted axially through the rings along with a ligament to cause the ligament to bulge radially outwardly and grip the rings.

22. The graft retaining system of claim 21 wherein the helical thread has a distal major diameter adjacent the distal end and a proximal major diameter adjacent the proximal end, the proximal major diameter being greater than the distal major diameter, the rings each being spaced a predetermined respective distance from the proximal end of the elongated members, the screw thread proximal major diameter being substantially contained in the space between the proximal end of the elongated members and the rings when the screw is fully inserted.

23. The graft retaining system of claim 21 wherein the screw further causes the proximal ends of the elongated members to deflect outwardly to tightly engage the bone.

24. A graft fixation implant for securing a graft in a bone tunnel, the implant comprising:
a cage having at least one elongated member situated in longitudinal alignment with an axis
at least one ring connected to the at least one elongated member; and
an interference screw sized to be inserted axially through the ring to compress the graft ligament against the cage and the bone tunnel wall.

25. A graft fixation implant for securing a graft in a bone tunnel having an inner tunnel wall, the implant comprising:
a cage body having a lumen for receiving a portion of the graft;
antirotation means for positively engaging the inner tunnel wall to resist rotation of the cage about the bone tunnel axis; and
means for compressing the graft radially outwardly into engagement with the cage body and inner tunnel wall.

26. An instrument for inserting a graft cage having a generally tubular cage body having an axis, a proximal end and a distal end, the cage having a fixed predetermined length, the cage body comprising a plurality of elongated, diametrically opposed, parallel rib members and at least one annular body member secured to the rib members, the annular body member having an axis coincident with the cage axis, the instrument comprising:
means for engaging the rib members to brace them against radial inward deflection; and
means for imparting an axial insertion force on the cage body.

27. The instrument of claim 26 wherein the means for engaging the rib members comprises a tubular sleeve having a sleeve wall with an inner surface, an outer surface, a proximal end, and a distal end, the sleeve wall including opposed longitudinal slots formed from the distal end toward the proximal end for receiving the rib members, each rib member further comprising a circumferential lip that engages the outer surface of the sleeve wall to prevent the rib member from deflecting radially inwardly.

28. The instrument of claim 27 further comprising a removable obturator that fits through the tubular sleeve and through the cage body, the obturator having a smooth distal tip to ease insertion of the cage into the bone tunnel.

29. The instrument of claim 28 wherein the obturator includes a longitudinal bore to permit it to be inserted over a guide wire.

30. An instrument for preparing a bone tunnel to receive a graft cage having a generally tubular cage body having an axis, a proximal end and a distal end, the cage having a fixed predetermined length, the cage body comprising a plurality of elongated, diametrically opposed, parallel rib members and at least one annular body member secured to the rib members, the annular body member having an axis coincident with the cage axis, the instrument comprising:
a shaft having a proximal end, a distal leading end, and an enlarged portion proximal to the distal leading end, the enlarged portion forming an enlarged counterbore in the bone tunnel upon being inserted into the bone tunnel, the counterbore formed being sized to receive the cage body.

31. The instrument of claim 30 further comprising rails projecting radially from the enlarged portion to form opposed channels adjacent the counterbore to receive the rib members.

32. A method for retaining a graft in a bone, the method comprising:
forming a tunnel in the bone;
inserting a first member into the bone tunnel, the first member having a plurality of longitudinally spaced closed rings;
inserting the graft through the rings; and
inserting a second member adjacent the graft to press the graft radially outwardly to cause it to bulge around the rings to fix the graft axially within the bone.

33. The method of claim 32 further comprising:
forming a counter bore adjacent the tunnel entrance prior to inserting the first member.

34. The method of claim 32 further comprising:
providing the first member with a plurality of longitudinal ribs connecting the rings, the ribs extending radially beyond the rings, the ribs being generally parallel to the tunnel axis;
forming channels inside the tunnel along the tunnel wall parallel to the tunnel axis;
inserting the first member with the ribs engaging the channels to resist rotation of the first member about the tunnel axis.

35. The method of claim 32 further comprising:
providing the first member with at least one ring axially aligned with the tunnel and a plurality of ribs connected to the at least one ring, the ribs being generally parallel to the tunnel axis;
engaging the ribs with an instrument to prevent radial inward deflection of the ribs prior to the second member being inserted.

36. The method of claim 35 further comprising:
inserting an obturator through the at least one ring to ease insertion of the first member, the obturator including a rounded leading end; and
withdrawing the obturator prior to inserting the graft.

37. A method of securing a graft ligament in a bone tunnel comprising:
providing a cage comprising at least one elongated member situated in longitudinal alignment with an axis, at least one ring connected to the at least one elongated member;
forming a bone tunnel;
inserting the cage into the bone tunnel;
inserting an end of a graft axially through the ring;
providing an interference screw; and
inserting the interference screw axially through the ring to compress the graft ligament against the ligament fixation implant and the bone tunnel wall.

38. A method of securing a graft ligament in a bone tunnel having an inner tunnel wall, the method comprising:
providing a cage including antirotation means for positively engaging the inner tunnel wall to resist rotation of the cage about the bone tunnel axis;
forming a bone tunnel;
inserting the cage into the bone tunnel with the antirotation means engaging the inner tunnel wall;
inserting an end of a graft axially through the cage;
providing an interference screw; and
inserting the interference screw axially through the ring means to compress the graft ligament against the cage and the bone tunnel wall.

39. The method of claim 38 wherein the antirotation means comprises a plurality of elongated members extending radially outwardly to engage a plurality of longitudinal channels formed along the inner tunnel wall, the method further comprising:
forming a plurality of longitudinal channels along the inner tunnel wall; and
engaging the plurality of elongated members with the plurality of longitudinal channels.
